(19) United States
(12) Patent Application Publication
(10) Pub. No.: US 2018/0374561 A1
(43) Pub. Date: Dec. 27, 2018

(54) SYSTEM FOR AUTOMATICALLY OBTAINING TREATMENT RECORD

(71) Applicant: SHENGHUO TECHNOLOGY LIMITED, Taipai City (TW)

(72) Inventor: Richard Jean Marie BINIER, Taipei City (TW)

(21) Appl. No.: 16/060,365

(22) PCT Filed: Dec. 7, 2015
(86) PCT No.: PCT/CN2015/096575

§ 371 Date: Jun. 7, 2018

(51) Int. Cl. G16H 10/60; G16H 20/17; G16H 40/67; A61B 5/145; G06F 17/30

(52) U.S. Cl. CPC G16H 10/60; G16H 20/17; G06F 17/30861; A61B 5/14532; G16H 40/67

(57) ABSTRACT

A system for automatically obtaining a treatment record, comprising including a measuring device configured to measure a physiological parameter and configured to generate a state information and a first physiological parameter value for each measurement, wherein the measuring device includes at least one display screen, a database configured to store a treatment record after each measurement, wherein the treatment record includes a dosage value and a second physiological parameter value after taking the dosage, a computing device, connected to the measuring device and to the database, configured to obtain the treatment record from the database according to the state information and the first physiological parameter value wherein the computing device calculates a times of use ratio of the dosage from the obtained treatment record and calculates an average value of at least two of a plurality of the second physiological parameter values after taking the same amount of dosage; and wherein the measuring device displays, through the display screen, the dosage, the times of use ratio corresponding to each of the dosages, and the average value of the at least two of the plurality of second physiological parameter values of each of the dosages.

### TECHNICAL FIELD

The disclosure relates to a system for automatically obtaining a treatment record, more particularly, to a system for a patient to obtain a treatment record after measuring physiological parameters.

### BACKGROUND

Injection pens, such as insulin injection pens, are provided for patients to perform self-injection at a selected dosage, such that the patients with chronic diseases can perform self-treatment at home. With respect to chronic diseases (e.g., diabetes) management, it is important that a historical information of when a dosage has been taken is available or accessible to the patients. For example, diabetic patients need to know how many dosages of insulin have been injected and the time of the injection, in order to determine the necessary dosage for the next injection accounting for occasions such as before or after consuming a meal. Therefore, it is important to provide the diabetic patient with a method for recording their injection records.

Injection records may be recorded on a remote device (e.g., smartphone) by wired or wireless communications, and the injection records may be browsed on the remote device. However, with the treatment records being accumulated gradually, patients are likely to be confused by the large volume of treatment records, which could result in misjudgment of dosage for the next injection, which would result in risk to the patient. Furthermore, it may be inconvenient for patients to go through the large volume of treatment records on the remote device before each injection.

### SUMMARY

A brief summary of various embodiments is presented below. Embodiments address a system for automatically obtaining a treatment record.

Some simplifications and omissions may be made in the following summary, which is intended to highlight and introduce some aspects of the various example embodiments, but not to limit the scope of the disclosure.

Detailed descriptions of example embodiments adequate to allow those of ordinary skill in the art to make and use the inventive concepts will follow in later sections.

Various embodiments described herein relate to a system for automatically obtaining a treatment record, including a measuring device configured to measure a physiological parameter and configured to generate a state information and a first physiological parameter value for each measurement, wherein the measuring device includes at least one display screen, a database configured to store a treatment record after each measurement, wherein the treatment record includes a dosage value and a second physiological parameter value after taking the dosage, a computing device, connected to the measuring device and to the database, configured to obtain the treatment record from the database according to the state information and the first physiological parameter value wherein the computing device calculates a times of use ratio of the dosage from the obtained treatment record and calculates an average value of at least two of a plurality of the second physiological parameter values after taking the same amount of dosage and wherein the measuring device displays, through the display screen, the dosage, the times of use ratio corresponding to each of the dosages, and the average value of the at least two of the plurality of second physiological parameter values of each of the dosages.

In an embodiment of the present disclosure, the times of use ratio is the percentage of the times of use of the each of the dosages used among the total times of use of the dosage.

In an embodiment of the present disclosure, the measuring device includes an input button for the patient to input to generate the state information before or after a meal.

In an embodiment of the present disclosure, the computing device communicates with the measuring device through Bluetooth or Near Field Communication (NFC) technology.

In an embodiment of the present disclosure, the computing device communicates with the database through a wireless network.

In an embodiment of the present disclosure, the measuring device is a blood glucose meter (“BGM”).

In an embodiment of the present disclosure, the first physiological parameter value and the second physiological parameter value are a blood sugar value.

In an embodiment of the present disclosure, the computing device is a smartphone.

### BRIEF DESCRIPTION OF THE DRAWINGS

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the disclosure. Where technical features in the figures, detailed description or any claim are followed by references signs, the reference signs have been included for the sole purpose of increasing the intelligibility of the figures, detailed description, and/or claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. In the figures:

FIG. 1 illustrates a schematic view of a system for automatically obtaining a treatment record according to an embodiment of the present disclosure;

FIG. 2 illustrates a schematic view of a treatment record obtained automatically according to an embodiment of the present disclosure;

FIG. 3 illustrates a schematic view of the result of a computing device of the system for automatically obtaining a treatment record according to an embodiment of the present embodiment, and

FIGS. 4A-4H illustrate schematic views of how to use the system for automatically obtaining a treatment record according to an embodiment of the present disclosure.

### DETAILED DESCRIPTION

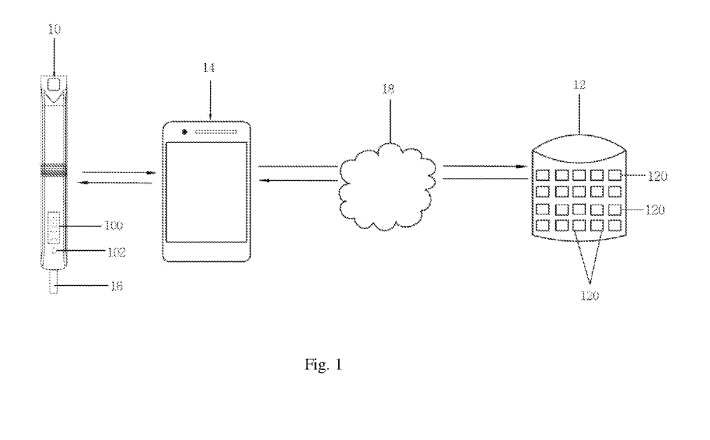

FIG. 1 illustrates a schematic view of a system 100 for automatically obtaining a treatment record according to an embodiment of the present disclosure.

The system 100 for automatically obtaining a treatment record includes a measuring device 10, a database 12 and a computing device 14. The measuring device 10 is configured to measure a physiological parameter of a patient, and the measuring device 10 includes a display screen 100.

For example, the measuring device 10 may be a blood glucose meter (“BGM”) for measuring a physiological parameter, such as a diabetic patient's blood glucose level (“BG level”).

The state information and the first physiological parameter value are generated and measured after a test paper 16 is placed into the measuring device 10, in which the first physiological parameter value may be the BG level of the patient.

The measuring device 10 may further include an input button 102, in which the input button 102 is provided for the patient to input and generate the state information before or after a meal. For example, when the input button 102 is pressed once, the measuring device 10 determines that the measurement is before a meal, and the time of the measurement is also recorded which is collectively recorded as the state information. If the input button 102 is pressed twice, the measuring device 10 determines that the measurement is after a meal, as well as the time of the measurement being recorded which is collectively recorded as the state information.

The database 12 stores a treatment record 120 of the measurement of the patient. The computing device 14 then obtains the treatment record 120 from the database 12 according to the recorded state information and the first physiological parameter value.

The computing device 14 may communicate with the measuring device 10 through Bluetooth or any near field communication (“NFC”) communication method, and may communicate with the database 12 through a wireless network 18. Moreover, the computing device 14 may connect to a smartphone.

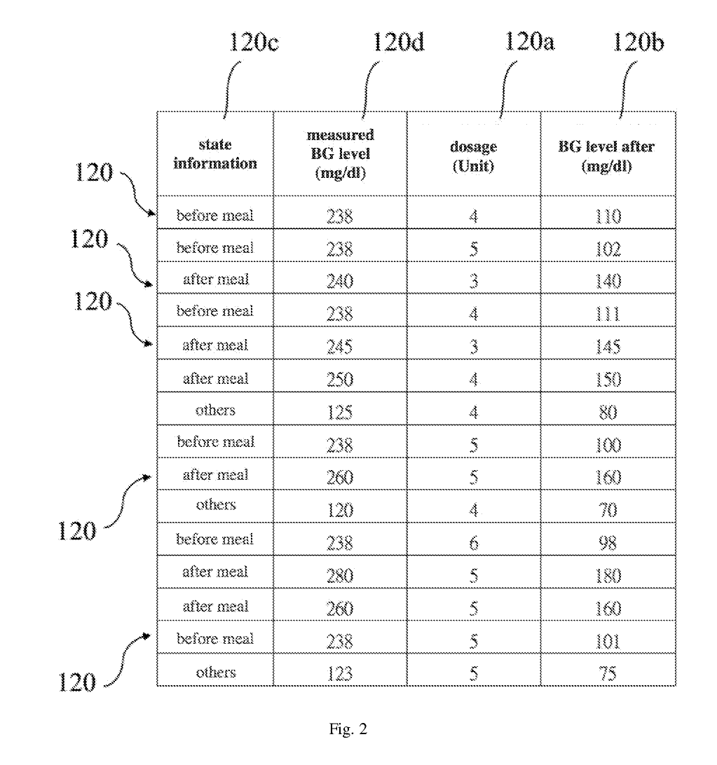

FIG. 2 illustrates a schematic view of a treatment record obtained automatically according to an embodiment of the present disclosure. FIG. 2 illustrates multiple treatment records 120, where the treatment records 120 may be stored in the database shown in FIG. 1.

Each treatment record 120 includes at least a dosage 120a and a second physiological parameter value after taking the dosage. The second physiological parameter value may be a BG level.

The treatment record 120 may further include state information 120c and a first physiological parameter value 120c before taking the dosage. In the present embodiment, the state information 120c may be set as before or after a meal, or other time-related items, and the first physiological parameter value is a BG level.

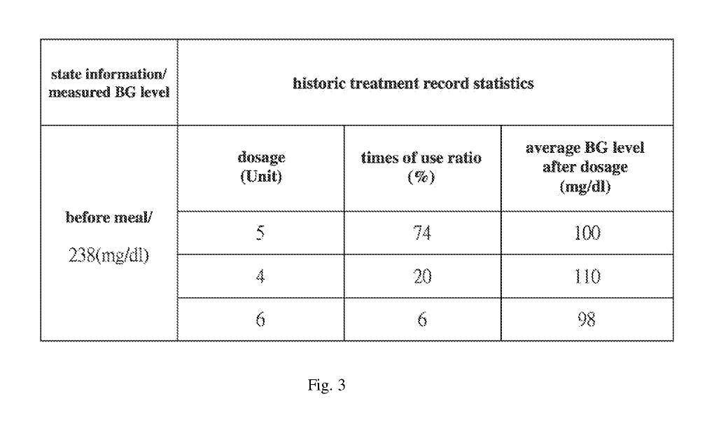

FIG. 3 illustrates a schematic view of the result of a computing device of the system for automatically obtaining a treatment record according to an embodiment of the present embodiment.

The computing device 14 may, according to the treatment record 120 obtained from the database 12, calculate the times of use ratio for different dosage.

The times of use ratio being the percentage of the number of times the different dosages are used over the total times of use of the dosages.

For example, as illustrated in FIGS. 2 and 3, by taking the state information 120c showing the first physiological parameter value 120d to be 238 (mg/dl), the computing device 14 may obtain the corresponding multiple treatment records 120 from the database 12, where the state information 120c indicates that all first physiological parameter values 120d which are 238 mg/dl where the state information is BG level before a meal.

The treatment records 120 that meet the above measuring requirements are six records, in which four units of dosage occurs two times, five units of dosage occurs three times and six units of dosage occurs one time.

The computing device 14 calculates the respective percentages of the five units dosage, the four units dosage and the six units dosage among the total six records. The respective percentages are 50%, 33% and 17%.

Moreover, the computing device 14 may, from the treatment record 12 obtained from the database, calculate an average value of multiple second physiological parameter values 120b of the patient after taking the same amount of dosage.

Therefore, the computing device 14 may calculate the average value of the multiple BG levels after the patient takes the five unit dosage, so as to obtain the average BG level, and the average BG level is equal to (102+100+101)/3=100 (mg/dl).

The average BG levels of the patient after respectively taking four units dosage and five units dosage calculated by the computing device 14 are 110 (mg/dl) and 98 (mg/dl), respectively.

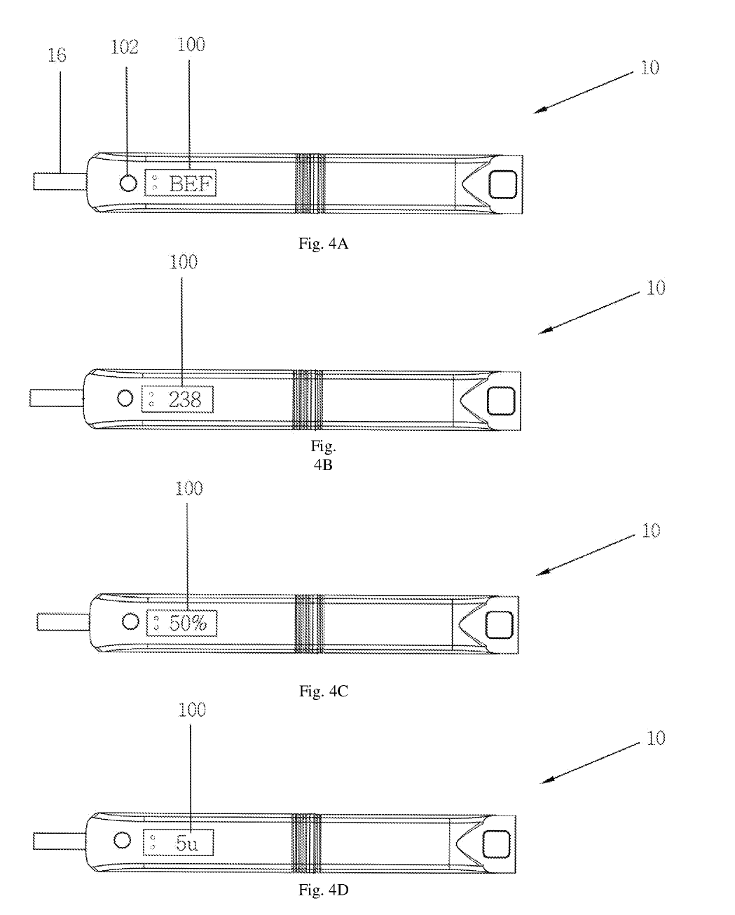

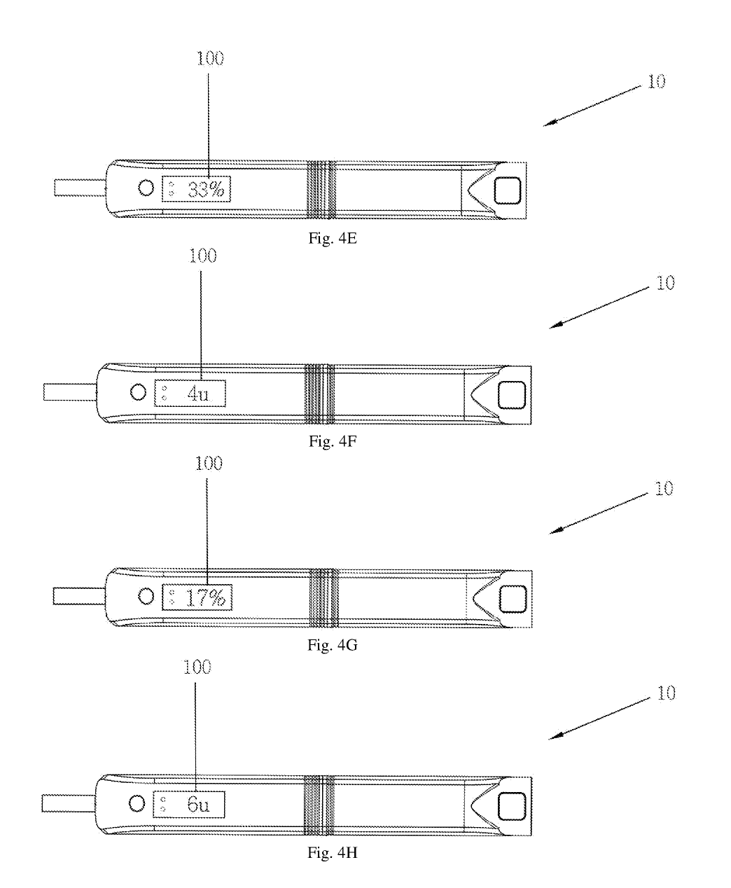

FIGS. 4A-4H are schematic views of how to use the system for automatically obtaining a treatment record according to an embodiment of the present disclosure.

The measuring device 10 can display, through the display screen 100, each of the dosages 120a, the times of use ratio corresponding to each of the dosages 120a (as shown in FIG. 3), and the average value corresponding to the multiple second physiological parameter values of each of the dosages 120a (as shown in FIG. 3).

The patient can press the input button 102 of the measuring device 12 once, and the display screen 100 will then display “BEF,” which indicates that the BG level before a meal is about to be measured. When the patient inserts the testing paper 16 with a blood sample into the measuring device 10, assuming that the measuring device 10 detects the BG level of the patient to be 238 (mg/dl), the measuring device 10 then transmits the state information to the computing device 14 through Bluetooth or other NFC method, the computing device 14 then obtains from the database the treatment record 120 with the same state information and the same BG level.

The computing device 14 may calculate the times of use ratio of different dosages in the obtained treatment record 120. For example, the computing device 14 calculates the percentages of the five units dosage, the four units dosage and the six units dosage of the total times of dosage, which are 50%, 33% and 17%, respectively (as shown in FIG. 3).

Therefore, in FIGS. 4C and 4D, the message of “50%” and “5u” are displayed in sequence on the display screen 100, indicating that the percentage of times of use of the five units dosage is 50%.

In FIGS. 4E and 4F, the message of “33%” and “4u” are displayed in sequence on the display screen 100, indicating that the percentage of times of use of the four units dosage is 33%.

In FIGS. 4H and 4H, the message of “17%” and “6u” are displayed in sequence on the display screen 100, indicating that the percentage of times of use of the six units dosage is 17%.

The system for automatically obtaining a treatment record according to the present disclosure analyzes a previous treatment record according to a current physiological parameter value of a patient, informs the patient of the analytical result and helps the patient to determine how much dosage of a drug is appropriate to be injected in an another injection.

As described above, optimal embodiments of the present disclosure have been disclosed in the drawings and the present specification. In this case, although specific terms have been used, those terms are merely intended to describe the present disclosure and are not intended to limit the meanings and the scope of the present disclosure as disclosed in the accompanying claims. Therefore, those skilled in the art will appreciate that various modifications and other equivalent embodiments are also possible given the above description. Therefore, the technical scope of the present disclosure should be defined by the technical spirit of the accompanying claims.

1. A system for automatically obtaining a treatment record, comprising:
a measuring device configured to measure a physiological parameter and configured to generate a state information and a first physiological parameter value for each measurement, wherein the measuring device includes at least one display screen;
a database configured to store a treatment record after each measurement, wherein the treatment record includes a dosage value and a second physiological parameter value after taking the dosage;
a computing device, connected to the measuring device and to the database, configured to obtain the treatment record from the database according to the state information and the first physiological parameter value;
wherein the computing device calculates a times of use ratio of the dosage from the obtained treatment record; and calculates an average value of at least two of a plurality of the second physiological parameter values after taking the same amount of dosage; and
wherein the measuring device displays, through the display screen, the dosage, the times of use ratio corresponding to each of the dosages, and the average value of the at least two of the plurality of second physiological parameter values of each of the dosages.

2. The system according to claim 1, wherein the times of use ratio is the percentage of the times of use of the each of the dosages used among the total times of use of the dosage.

3. The system according to claim 1, wherein the measuring device includes an input button for the patient to input to generate the state information before or after a meal.

4. The system according to claim 1, wherein the computing device communicates with the measuring device through Bluetooth or Near Field Communication (NFC) technology.

5. The system according to claim 1, wherein the computing device communicates with the database through a wireless network.

6. The system according to claim 1, wherein the measuring device is a blood glucose meter (“BGM”).

7. The system according to claim 1, wherein the first physiological parameter value and the second physiological parameter value are a blood sugar value.

8. The system according to claim 1, wherein the computing device is a smartphone.
